(19) United States
(12) Patent Application Publication
(10) Pub. No.: US 2005/0209271 A1
(43) Pub. Date: Sep. 22, 2005

(54) Form of renzapride hydrochloride hydrate and uses thereof

(76) Inventors: Richard Michael John Palmer, Beckenham (GB); Nicholas Leslie Meyers, Wickhambrook (GB); John Knight, Grove (GB)

Correspondence Address: Patent Department;Choate, Hall & Stewart, 53 State Street, Exchange Place, Boston, MA, 02109, US

(21) Appl. No.: 11/016,091

(22) Filed: Dec. 17, 2004

(30) Foreign Application Priority Data
Dec. 18, 2003 (GB) GB 0329319.8

(51) Int. Cl. A61K 31/4745; C07D 453/02

(52) U.S. Cl. 514/305; 546/133

(57) ABSTRACT

The present invention provides a novel crystal form of renzapride hydrochloride hydrate. The invention further provides methods of preparing the same and uses thereof.

### CROSS REFERENCE TO RELATED APPLICATIONS

This application claims priority, under 35 U.S.C. §119 (a), to Patent Application serial number GB 0329319.8 filed Dec. 18, 2003, the contents of which are incorporated herein by reference.

### FIELD OF THE INVENTION

The present invention provides a novel crystal form of renzapride hydrochloride hydrate. The invention further provides methods of preparing the same and uses thereof.

### BACKGROUND OF THE INVENTION

EP-A-94742 discloses the substituted azabicyclo compound, (±)-4-amino-5-chloro-2-methoxy-N-(1-azabicyclo[3.3.1]non-4-yl)benzamide known by the generic name renzapride (also known as renzapride free base). In addition, EP-A-94742 provides general procedures for preparing the disclosed substituted azabicyclo compounds. Such compounds are useful in the treatment of disorders relating to impaired gastro-intestinal motility, such as retarded gastric emptying, dyspepsia, flatulence, oesophageal reflux, and peptic ulcer, in the treatment of emesis and disorders of the central nervous system.

The hydrochloride salt of renzapride (renzapride hydrochloride) is preferred over the free base because of its improved stability. EP-A-0239321 discloses the hydrate form of the hydrochloride salt of renzapride, which provides improved handling and stability characteristics over the anhydrous hydrochloride salt of renzapride.

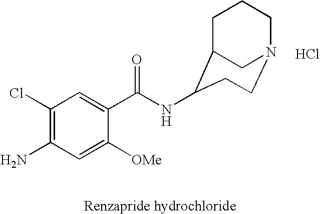

The 5-HT₄ receptor agonist and 5-HT_{2B} and 5-HT₃ receptor antagonist activities of renzapride hydrochloride hydrate make it an ideal candidate for treating various disorders associated with these receptors.

Accordingly, it would be desirable to provide renzapride in a form suitable for administration to a patient in the treatment of such disorders. In particular, it would be desirable for such compounds to exhibit additional characteristics such as good solubility, stability and ease of formulation, etc.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts the solid state ¹³C-NMR of Form II renzapride hydrochloride hydrate;

FIG. 2 depicts the solid state ¹³C-NMR of amorphous renzapride hydrochloride hydrate;

FIG. 3 depicts the DVS sorption plot of Form II renzapride hydrochloride hydrate;

FIG. 4 depicts the DVS sorption plot of Form II renzapride hydrochloride hydrate;

FIG. 5 depicts the DVS sorption plot of Form II renzapride hydrochloride hydrate;

FIG. 6 depicts the Isotherm map for Form II renzapride hydrochloride hydrate;

FIG. 7 depicts the DVS sorption plots and isotherm maps for Form II renzapride hydrochloride hydrate;

FIG. 8 depicts the DVS sorption plot of Form II renzapride hydrochloride hydrate;

FIG. 9 depicts the isotherm map for Form II renzapride hydrochloride hydrate;

FIG. 10 depicts the DVS sorption plot of amorphous renzapride hydrochloride hydrate;

FIG. 11 depicts the isotherm map for amorphous renzapride hydrochloride hydrate;

FIG. 12 depicts the IR spectrum of Form II renzapride hydrochloride hydrate;

FIG. 13 depicts an expanded region of the IR spectrum of Form II renzapride hydrochloride hydrate;

FIG. 14 depicts the IR spectrum of Form II renzapride hydrochloride hydrate;

FIG. 15 depicts an expanded region of the IR spectrum of Form II renzapride hydrochloride hydrate;

FIG. 16 depicts an expanded region of the IR spectrum of Form II renzapride hydrochloride hydrate;

FIG. 17 depicts the IR spectrum of Form II renzapride hydrochloride hydrate;

FIG. 18 depicts an expanded region of the IR spectrum of Form II renzapride hydrochloride hydrate;

FIG. 19 depicts the IR spectrum of Form II renzapride hydrochloride hydrate;

FIG. 20 depicts an expanded region of the IR spectrum of Form II renzapride hydrochloride hydrate;

FIG. 21 depicts the X-ray powder diffraction pattern for Form II renzapride hydrochloride hydrate;

FIG. 22 depicts the thermal analysis of Form II renzapride hydrochloride hydrate;

### DETAILED DESCRIPTION OF CERTAIN EMBODIMENTS OF THE INVENTION

General Description of Certain Aspects of the Invention:

A new crystalline form of renzapride hydrochloride hydrate has been identified, which provides improved properties for its use in treating various disorders. This new crystalline form has been designated Form II.

Accordingly, the first aspect of the present invention provides a method for preparing Form II renzapride hydrochloride hydrate comprising the steps of:
(a) providing renzapride;
(b) incubating renzapride in a solution of water and a water miscible solvent;
(c) adding concentrated hydrochloric acid to the renzapride solution; and
(d) isolating the resulting Form II renzapride hydrochloride hydrate.

In certain embodiments, the isolation at step (c) is performed by filtration.

In other embodiments, the water miscible solvent for the purposes of this invention is one or more of tetrahydrofuran (THF), acetone and/or an alcohol. In other embodiments, the alcohol is selected from one or more of methanol, ethanol, n-propanol, isopropanol, n-butanol or tert-butanol. In still other embodiments, the alcohol is ethanol.

According to one embodiment, the water/water miscible solvent solution contains from 3% to 15% water. According to another embodiment, the water/water miscible solvent solution contains from 5% to 10% water. According to yet another embodiment, the water/water miscible solvent solution contains from 8% water or above.

In certain embodiments, the incubation of renzapride in the water/water solvent solution is carried out with agitation, such as with stirring. The renzapride solution can be incubated at 20-25° C. However, the renzapride solution can be heated to allow the dissolution of renzapride in the water/water miscible solvent solution. In other embodiments, the renzapride solution is initially incubated at 20-25° C. for example for 15 to 30 minutes followed by incubation with heat, for example at reflux, to allow dissolution.

In other embodiment, after incubation of renzapride in the water/water miscible solvent solution, the solution is optionally filtered to remove any particulate material.

In certain embodiments, addition of the hydrochloric acid to the renzapride solution is carried out at 60-70° C. The temperature of the reaction mixture can then be reduced to room temperature, more preferably to 20-25° C. and incubation carried out for one or more periods of 1 to 2 hours. Optionally, the reaction mixture can additionally be incubated for one or more periods of 1 to 2 hours at 0-5° C.

In certain embodiments, the isolated Form II renzapride hydrochloride hydrate is dried in vacuo to reduce the solvent, e.g. ethanol, content to <3% and the resultant solid exposed to water, preferably purified water, in an enclosed area in order to modify the solvent content, e.g. the ethanol content, of the product to levels acceptable for commercial use while maintaining the final Form II product.

In other embodiments, the isolated Form II renzapride hydrochloride is exposed to water without the need to dry in vacuo, preferably the Form II renzapride hydrochloride can be exposed to purified water in an enclosed area in order to modify the solvent, e.g. ethanol content of the product from about 3% (w/w) to levels acceptable for commercial use. For the purposes of this invention, the level acceptable for commercial use is a level of 1% (w/w) solvent or less, preferably 0.1% (w/w) solvent or less.

General Methods of Providing the Present Compounds:

Renzapride can be prepared according to the methods described in EP-A-94742 and/or GB 0321091.1. As used herein, the term renzapride relates to the free base as illustrated below 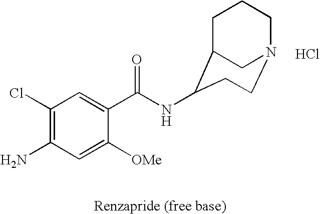
and renzapride hydrochloride relates to the hydrochloride salt of renzapride as illustrated below.

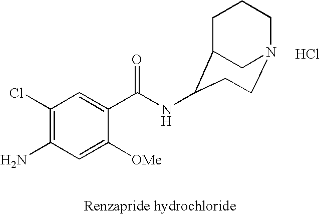

In certain embodiments, renzapride is prepared by the condensation of a substituted phenyl (11) with an amine (14) to give the condensation product (8). In particular, the substituted phenyl (11) can be activated, for example to the acid chloride (13) 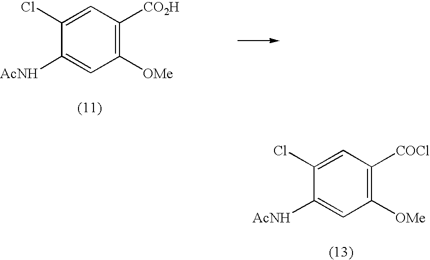
and the acid chloride (13) of compound (11) can be condensed with an amine (14) to give the condensation product (8), 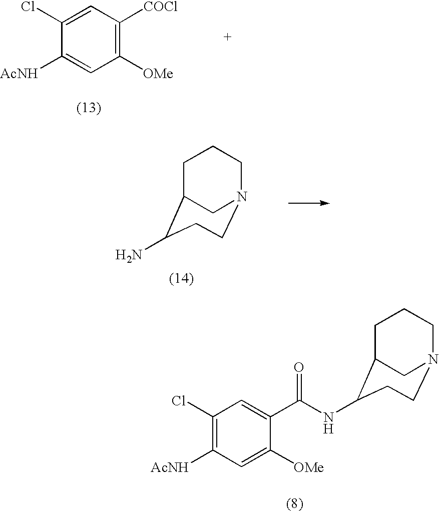
followed by the deprotection of compound (8) to provide renzapride.

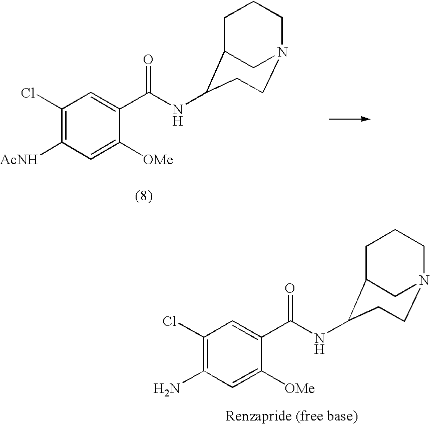

Compounds (11) and (14), as depicted above, are prepared by methods disclosed in EP-A-94742 and GB0321091.1.

In certain embodiments, renzapride is used directly in the process of the first aspect of the invention to provide Form II renzapride hydrochloride hydrate. The first aspect of the invention therefore provides a convenient one-step process for the production of Form II renzapride hydrochloride hydrate from renzapride.

The second aspect of the invention provides a process for the formation of Form II renzapride hydrochloride hydrate from renzapride hydrochloride, said process comprising the steps of:
(a) forming a saturated solution of renzapride hydrochloride in a solvent system comprising an organic solvent and from 3% to 30% water; and
(b) isolating Form II renzapride hydrochloride hydrate therefrom.

In certain embodiments of the second aspect of the invention, the Form II renzapride hydrochloride hydrate is isolated by crystallisation. Crystallisation of the Form II renzapride hydrochloride hydrate can be initiated by methods known in the art. According to one embodiment, Form II renzapride hydrochloride hydrate is crystallised from a solvent system by cooling the saturated solution of renzapride hydrochloride hydrate to 10° C. or less. In other embodiments, the saturated solution of renzapride hydrochloride hydrate is cooled to 0° C. or less. In still other embodiments, the saturated solution of renzapride hydrochloride hydrate is cooled to −5° C. or less. The cooling of the saturated solution of renzapride hydrochloride hydrate may be accompanied by stirring until crystallisation of the renzapride hydrochloride hydrate is complete or has provided the desired amount of crystalline renzapride hydrochloride hydrate.

It may be necessary to add a miscible organic solvent in which the renzapride hydrochloride hydrate is not soluble to facilitate crystallisation (referred to as the miscible non-solvent, for the purposes of this invention). Alternatively seed crystals of Form II renzapride hydrochloride hydrate may be added to the saturated solution of renzapride hydrochloride hydrate. The addition of such seed crystals can be used separately, simultaneously or sequentially with the use of cooling and/or stirring, and/or the addition of a miscible non-solvent.

In order to produce a saturated solution of renzapride hydrochloride, it may be necessary to warm the solution. Preferably, the renzapride hydrochloride solvent mixture is heated to reflux. The solution may be stirred or agitated to produce or aid production of the saturated solution.

For the purposes of this invention the solvent system may comprise one or more solvents which can solubilise renzapride hydrochloride and which are miscible with water. Preferably the solvents are one or more of ethanol, acetone, isopropyl alcohol, tertiary-butyl methylether (TBME), or THF, more preferably ethanol.

In certain embodiments, the crystallisation of renzapride hydrochloride hydrate is carried out in an aqueous ethanol solution more preferably a 20% aqueous ethanol solution.

Isolation of the Form II renzapride hydrochloride hydrate can be achieved by filtration. Any solvent residue remaining in the isolated product can be removed by washing the crystalline solid in an organic solvent. Preferably, the washing solvent is more volatile than the solvent residue and can itself be easily removed from the product. Examples of such solvents for the purposes of this invention include THF, n-heptane or toluene. Alternatively, the isolated product can be washed in a cold organic solution comprising from 4 to 25% water more preferably 8% water or above, such as 8% aqueous ethanol.

The product can be dried to remove any remaining solvent. Preferably, the drying does not reduce the percentage water of the product. However should such reduction in the percentage of water occur, the product should be rehydrated to produce the Form II renzapride hydrochloride hydrate. Methods for drying the product include the use of fluidised bed drying and air drying in an oven in the presence or absence of a vacuum. Preferably, the drying is carried out in an inert atmosphere such as a nitrogen atmosphere.

Solvent residue can further be removed by slurrying the product in an organic solvent. Again, the slurry solvent should be more volatile than the solvent residue so that it can be easily removed from the product. Examples of suitable slurrying solvents include TBME.

The third aspect of the invention provides a process for the formation of Form II renzapride hydrochloride hydrate comprising the steps of:
(a) slurrying renzapride hydrochloride in an organic solvent comprising 4 to 25% water; and
(b) isolating Form II renzapride hydrochloride hydrate therefrom.

In certain embodiments, the organic solvent is miscible with water and is selected from one or more of ethanol, acetone, isopropyl alcohol, TBME or THF. In other embodiments, the solvent is ethanol.

According to one embodiment, the organic solvent used in step (a) comprises 6 to 10% water. In other embodiments, said organic solvent used in step (a) comprises 8% water or above. In still other embodiments, said organic solvent used in step (a) comprises 8% water. In other embodiments, said organic solvent is ethanol.

Renzapride hydrochloride as discussed in the second and third aspects of the invention can be obtained according to the processes set out in EP 0239321. Renzapride hydrochloride can be provided in a hydrated or non-hydrated form for the second and third aspects of the invention.

In particular, renzapride (in its free base form) is dissolved in a suitable solvent, preferably ethanol, and a solution of hydrochloric acid in a suitable solvent, preferably ethanol, is added, allowing the product to precipitate.

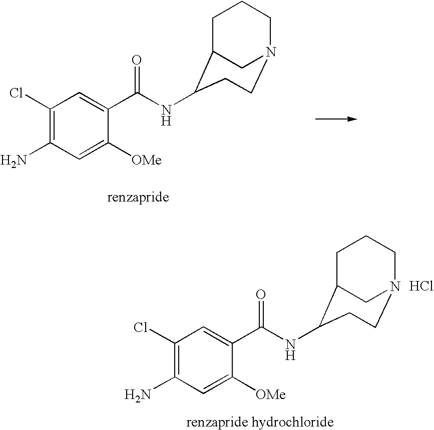

The fourth aspect of the invention provides crystalline Form II renzapride hydrochloride hydrate comprising two moles of water per mole of renzapride hydrochloride at a level of 75% Form II or above.

In certain embodiments, Form II renzapride hydrochloride hydrate is provided at a level of 80% or above. In other embodiments, Form II renzapride hydrochloride hydrate is provided at 90% or above. In still other embodiments, Form II renzapride hydrochloride hydrate is provided at 95% or above.

Form II renzapride hydrochloride hydrate is provided as the dihydrate form. In certain embodiments, the Form II renzapride hydrochloride hydrate of the present invention contains from 8.3 to 9.8% water. In other embodiments, the Form II renzapride hydrochloride hydrate of the present invention contains from 8.5 to 9.6% water. In still other embodiments, the Form II renzapride hydrochloride hydrate of the present invention contains 9.0% water.

Without wishing to be bound by any particular scientific theory, it is proposed and studies indicate that the water is bound within the crystal structure of the renzapride molecules and is not loosely associated with the molecules.

The provision of renzapride as the Form II renzapride hydrochloride hydrate provides a number of advantageous properties over those observed for amorphous renzapride hydrochloride hydrate. The advantageous properties of this crystalline form include improved stability to atmospheric water or moisture, improved filtering and improved drying. It will be appreciated by a person skilled in the art that the properties of Form II renzapride hydrochloride hydrate make this form of renzapride particularly preferred for use as a medicament. In particular, Form II renzapride hydrochloride hydrate shows good stability with respect to moisture and can therefore be stored on a long-term basis without deterioration. In particular, Form II renzapride hydrochloride hydrate can be stored on a long term basis without a significant change in the water content of the stored medicament.

It has been found that Form II renzapride hydrochloride hydrate exhibits a narrow particle size distribution. This provides a form of renzapride hydrochloride hydrate, which allows the production of homogeneous formulation blends, especially at low dosage strengths. The Form II renzapride hydrochloride hydrate may additionally not require screening during blending of the material thereby allowing more efficient formulation. Form II renzapride hydrochloride hydrate additionally exhibits improved filtering characteristics compared with the amorphous form.

Furthermore the provision of Form II renzapride hydrochloride hydrate allows the formation of a uniform blend without the need for size reduction, filtering etc. This allows more effective handling and formulation of renzapride. Furthermore, the improved water stability of Form II renzapride hydrochloride hydrate allows more efficient drying of the active ingredients, facilitating its formulation into for example a capsule or tablet form.

Form II is therefore particularly preferred in the preparation of a composition comprising renzapride as its narrow particle size distribution allows Form II to be used for low strength capsules or tablets without the need for milling or micronisation.

Form II renzapride hydrochloride hydrate shows consistent behaviour when Form II is exposed to moisture. This form can therefore be stored on a long-term basis. Furthermore the material will behave in a predictable fashion during dispensing and manufacture.

The improved properties of Form II renzapride hydrochloride hydrate mean that the formulation of Form II renzapride hydrochloride hydrate into a dosage form such as a tablet is more time, energy and cost efficient that the formulation of amorphous renzapride hydrochloride hydrate. Furthermore, both Form II renzapride hydrochloride hydrate and formulations thereof can be stored on a long-term basis due to the stability of Form II renzapride hydrochloride hydrate with respect to moisture.

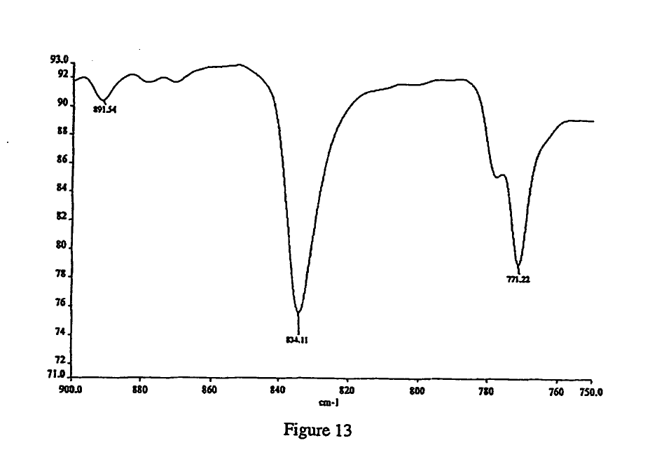

Form II renzapride hydrochloride hydrate can be characterised by its Infra-Red spectrum, wherein Form II renzapride hydrochloride hydrate exhibits a diagnostic peak at 835±1.5 cm⁻¹. Accordingly, the present invention provides a method for the identification of Form II renzapride hydrochloride hydrate in a sample characterised by carrying out infra-red spectroscopy on a sample of renzapride hydrochloride hydrate and monitoring for the diagnostic peak at 835±1.5 cm⁻¹ (as illustrated for example in FIG. 13).

Pharmaceutically Acceptable Compositions

The fifth aspect of the invention relates to a pharmaceutical composition comprising Form II renzapride hydrochloride hydrate as defined herein in the fourth aspect of the invention and a pharmaceutically acceptable carrier, adjuvant or vehicle.

Suitable carriers and/or diluents are well known in the art and include pharmaceutical grade starch, mannitol, lactose, magnesium stearate, sodium saccharin, talcum, cellulose, glucose, sucrose (or other sugar), magnesium carbonate, gelatin, oil, alcohol, detergents, emulsifiers or water (preferably sterile). The composition may be a mixed preparation of a composition or may be a combined preparation for simultaneous, separate or sequential use (including administration).

The compounds according to the invention for use in the aforementioned indications may be administered by any convenient method, for example by oral (including by inhalation), parenteral, mucosal (e.g. buccal, sublingual, nasal), vaginal, rectal or transdermal administration and the compositions adapted accordingly.

Form II renzapride hydrochloride hydrate according to the present invention can be provided in a delayed release composition. This delayed release composition comprises Form II renzapride hydrochloride hydrate in combination with a delayed release component. This composition allows targeted release of Form II renzapride hydrochloride hydrate into the lower gastrointestinal tract for example into the small intestine, the large intestine, the colon and/or the rectum. The delayed release composition may comprise Form II renzapride hydrochloride hydrate and an enteric or pH dependent coating such as cellulose acetate phthalates and other phthalates (e.g. polyvinyl acetate phthalate, methacrylates (Eudragits)). Alternatively, the delayed release composition may provide controlled release to the small intestine and/or colon by the provision of pH sensitive methacrylate coatings, pH sensitive polymeric microspheres, or polymers which undergo degradation by hydrolysis. The delayed release composition can be formulated with hydrophobic or gelling excipients or coatings. Colonic delivery can further be provided by coatings which are digested by bacterial enzymes such as amylose or pectin, by pH dependent polymers, by hydrogel plugs swelling with time (Pulsincap), by time dependent hydrogel coatings and/or by acrylic acid linked to azoaromatic bonds coatings.

For oral administration, the compounds can be formulated as liquids or solids, for example solutions, syrups, suspensions, emulsions, tablets, capsules, lozenges, dry powder and/or granules.

A liquid formulation will generally consist of a suspension or solution of the compound or physiologically acceptable salt in a suitable aqueous or non-aqueous liquid carrier(s) for example water, ethanol, glycerol, polyethylene glycol or an oil. The formulation may also contain a suspending agent, preservative, flavouring or colouring agent.

A composition in the form of a tablet can be prepared using any suitable pharmaceutical carrier(s) routinely used for preparing solid formulations. Examples of such carriers include magnesium stearate, starch, lactose, sucrose and microcrystalline cellulose.

A composition in the form of a capsule can be prepared using routine encapsulation procedures. For example, powders, granules or pellets containing the active ingredient can be prepared using standard carriers and then filled into a capsule, for example a hard gelatin capsule, a HPMC capsule, a soft gelatin capsule etc; alternatively, a dispersion or suspension can be prepared using any suitable pharmaceutical carrier(s), for example aqueous gums, celluloses, silicates or oils and the dispersion or suspension then filled into a soft gelatin capsule.

Compositions for oral administration may be designed to protect the active ingredient against degradation as it passes through the alimentary tract, for example by an outer coating of the formulation on a tablet or capsule.

Typical parenteral compositions consist of a solution or suspension of the compound or physiologically acceptable salt in a sterile aqueous carrier or non-aqueous or parenterally acceptable oil, for example polyethylene glycol, polyvinyl pyrrolidone, lecithin, arachis oil or sesame oil. Alternatively, the solution can be lyophilised and then reconstituted with a suitable solvent just prior to administration.

Compositions for nasal or oral administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively the sealed container may be a unitary dispensing device such as a single dose nasal inhaler or an aerosol dispenser fitted with a metering valve which is intended for disposal once the contents of the container have been exhausted. Where the dosage form comprises an aerosol dispenser, it will contain a pharmaceutically acceptable propellant. The aerosol dosage forms can also take the form of a pump-atomiser.

Compositions suitable for buccal or sublingual administration include tablets, lozenges and pastilles, wherein the active ingredient is formulated with a carrier such as sugar and acacia, tragacanth, or gelatin and glycerin.

Compositions for rectal or vaginal administration are conveniently in the form of suppositories (containing a conventional suppository base such as cocoa butter), pessaries, vaginal tabs, foams or enemas.

Compositions suitable for transdermal administration include ointments, gels and patches, and injections, including powder injections.

Conveniently the composition is in unit dose form such as a tablet, capsule or ampoule.

The compositions may contain from 0.1% to 99% (w/w) preferably from 0.1-60% (w/w), more preferably 0.2-20% by weight and most preferably 0.25 to 12% (w/w) of the Form II renzapride hydrochloride hydrate, depending on the method of administration.

Uses and Methods

The sixth aspect of the invention relates to Form II renzapride hydrochloride hydrate as defined in the fourth aspect of the invention, or a pharmaceutical composition thereof as defined in the fifth aspect of the invention, for treating and/or preventing a disorder relating to impaired gastro-intestinal motility and/or abdominal pain. Accordingly, the present invention provides a method of treating, preventing, or lessening the severity of a disorder relating to impaired gastro-intestinal motility and/or abdominal pain in a patient in need thereof, wherein said method comprises the step of administering to said patient Form II renzapride hydrochloride hydrate, or a composition thereof.

For the purposes of the present invention, gastro-intestinal includes the oesophagus, the stomach, the small intestine and the large intestine (including the colon and the rectum). Form II renzapride hydrochloride hydrate may generally be used in the treatment of disorders relating to impaired gastro-intestinal motility. The disorders include one or more of irritable bowel syndrome, retarded or delayed gastric emptying, dyspepsia, oesophageal reflux, peptic ulcer, flatulence, impaired evacuation, constipation, diabetic neuropathy, functional abdominal bloating, gastroparesis or abdominal pain.

Form II renzapride hydrochloride hydrate can also be used in the treatment of symptoms associated with such disorders including abdominal pain and/or discomfort, abdominal bloating, an abnormality in stool consistency, an abnormality in frequency of stool passage, a feeling of incomplete emptying, feelings of urgency and passage of mucus. It may also be used in the treatment of emesis and/or the treatment of disorders of the central nervous system such as psychosis. Preferably Form II renzapride hydrochloride hydrate is used for the treatment of irritable bowel syndrome, more preferably constipation-predominant, diarrhoea-predominant or alternating (mixed-symptom) irritable bowel syndrome.

It has been found that renzapride hydrochloride hydrate is additionally effective in the treatment of irritable bowel syndrome (IBS), constipation, gastroparesis and abdominal pain and discomfort. Accordingly, another embodiment of the present invention provides a method of treating, preventing, or lessening the severity of irritable bowel syndrome (IBS), constipation, gastroparesis and abdominal pain and discomfort in a patient in need thereof wherein said method comprises the step of administering to said patient Form II renzapride hydrochloride, hydrate or a composition thereof.

The amount of Form II renzapride hydrochloride hydrate effective to treat a disorder as set out above depends on the nature and severity of the disorder being treated and the weight of the patient in need thereof. However, a single unit dose for a 70 kg adult will normally contain 0.01 to 100 mg, for example 0.1 to 50 mg, preferably 0.5 to 16 mg of the compound of the invention per day. Unit doses may be administered once or more than once a day, for example, 2, 3 or 4 times a day, usually 1 to 3 times a day, more preferably 1 or 2 times per day. It will be appreciated that the dose ranges set out above provided guidance for the administration of Form II renzapride hydrochloride hydrate to an adult. The amount to be administered to for example, an infant or a baby can be determined by a medical practitioner or person skilled in the art and can be lower or the same as that administered to an adult. The unit dose is preferably provided in the form of a capsule or a tablet.

The seventh aspect of the invention relates to a method of treating a disorder relating to impaired gastro-intestinal motility comprising and administering to a subject in need thereof. Form II renzapride hydrochloride hydrate as defined in the fourth aspect of the invention or a pharmaceutical composition as defined in the fifth aspect of the invention.

All preferred features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXAMPLES

### Example 1

As described above, renzapride and renzapride hydrochloride are obtained using the methods set out in EP-A-0094742, EP-A-0239321 and GB 0321091.1.

### Example 2

### Preparation of Form II Renzapride Hydrochloride Hydrate

Salt Formation Procedure:

The free base of renzapride (1 wt) was suspended in 8% aqueous ethanol (5 vol; made up from 0.4 vol. water and 4.6 vol. absolute ethanol) and stirred at 20-25° C. for 15-30 minutes. The mixture was heated to reflux and held at reflux until dissolution was achieved (up to 70 minutes expected) and then cooled to 60-65° C. and clarified through pre-heated lines and filter (1 μm) to remove any particulate material. The lines and filter were rinsed with hot (60-65°) 8% aqueous ethanol (1 vol. made from 0.08 vol. water 0.92 vol. absolute ethanol). The solution of free base was then treated with concentrated hydrochloric acid (1.05 mol. equiv.) maintaining the internal temperature in the range 60-70° C. The resultant mixture was cooled to 20-25° C. and aged in this range for 1-2 hours. The resultant slurry was further cooled to 0.5° C. and aged for a further 1-2 hours. A sample of the slurry was removed, filtered and the solid checked by IR to ensure it is Form II. The slurry may be aged for further 1-2 hours periods as necessary to ensure the material is Form II ahead of isolation.

The product was isolated by filtration and the filter cake washed by displacement with cool (0-5° C.) 8% aqueous ethanol (1 vol., made from 0.08 vol. water and 0.92 vol. absolute ethanol) and then pulled down on the filter for 3-4 hours. The solid was transferred to trays and stood in an enclosed area in the presence of purified water until the ethanol content was 1% (w/w) or less.

Recrystallization Procedure:

Renzapride hydrochloride (1 wt) was treated with 20% aqueous ethanol (3 vol., made from 0.6 vol. water and 2.4 vol. absolute ethanol). The stirred mixture was heated to reflux and held at reflux until dissolution is achieved (up to 70 minutes expected) and then cooled to 60-65° C. and clarified through pre-heated lines and filter (1 μm) to remove any particulate material. The lines and filter were rinsed with hot (60-65° C.) ethanol (4.5 vol.), maintaining the temperature of the renzapride hydrochloride solution at 60-65° C. throughout.

The resultant mixture was cooled to 20-25° C. and aged in this range for 1-2 hours. The resultant slurry was further cooled to 0-5° C. and aged for a further 1-2 hours. A sample of the slurry was removed, filtered and the solid checked by IR to ensure it is Form II. The slurry may be aged for further 1-2 hours periods as necessary to ensure the material is Form II ahead of isolation.

The product was isolated by filtration and the filter cake washed by displacement with cool (0-5° C.) 8% aqueous ethanol (1 vol., made from 0.08 vol. water and 0.92 vol. absolute ethanol) and then pulled down on the filter for 3-4 hours. The solid was transferred to trays and stood in an enclosed area in the presence of purified water until the ethanol content was 1% (w/w) or less.

Slurry Procedure:

The renzapride hydrochloride (1 wt) was treated with 8% aqueous ethanol (5 vol.) and the suspension stirred and cooled to 0-5° C. and once in range, stirred at 0-5° C. for 2-3 hours. A sample of the slurry was removed, filtered and the solid checked by IR to ensure it is Form II. The slurry may be aged for further 1-2 hour periods as necessary to ensure the material is Form II ahead of isolation.

The product was isolated by filtration and the filter cake washed by displacement with cool (0-5° C.) 8% aqueous ethanol (1 vol., made from 0.08 vol. water and 0.92 vol. absolute ethanol) and then pulled down on the filter for 3-4 hours. The solid was transferred to trays and stood in an enclosed area in the presence of purified water until the ethanol content was 1% (w/w) or less.

### Example 2

### Manufacture of Amorphous Renzapride Hydrochloride Hydrate

Amorphous renzapride hydrochloride hydrate was prepared according to EP-A-0094742 and EP-A-0239321. As used herein, the term “amorphous” refers to a sample of renzapride hydrochloride hydrate comprising non-crystalline and crystalline material, wherein said crystalline material may be present in a mixture of one or more forms.

### Example 3

### Comparison of Form II Renzapride Hydrochloride Hydrate and Amorphous Renzapride Hydrochloride Hydrate

Solid State ¹³C-NMR:

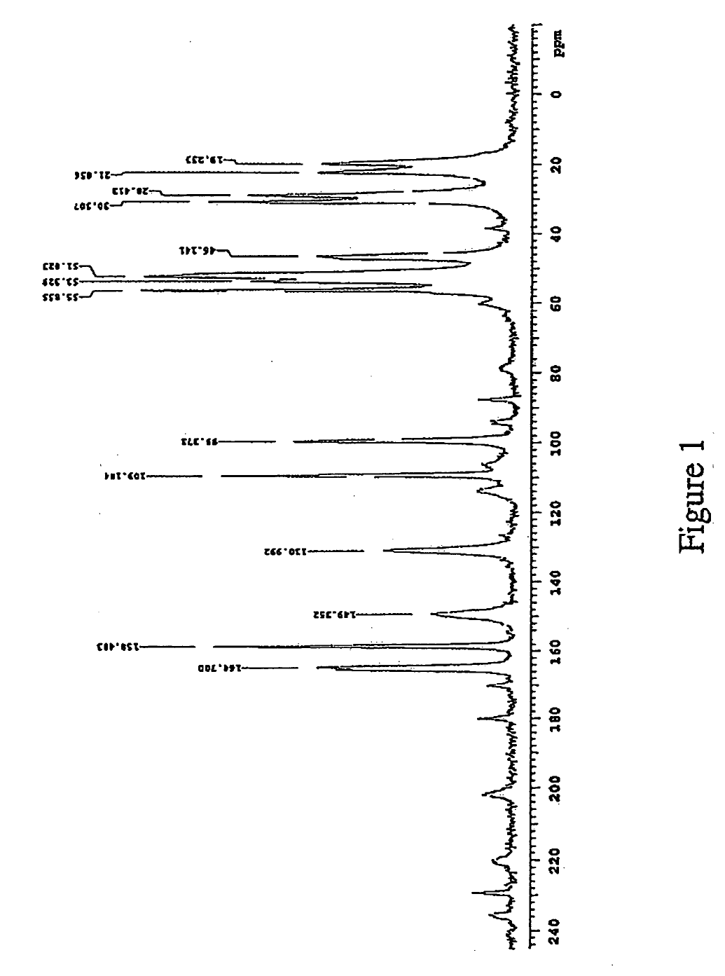

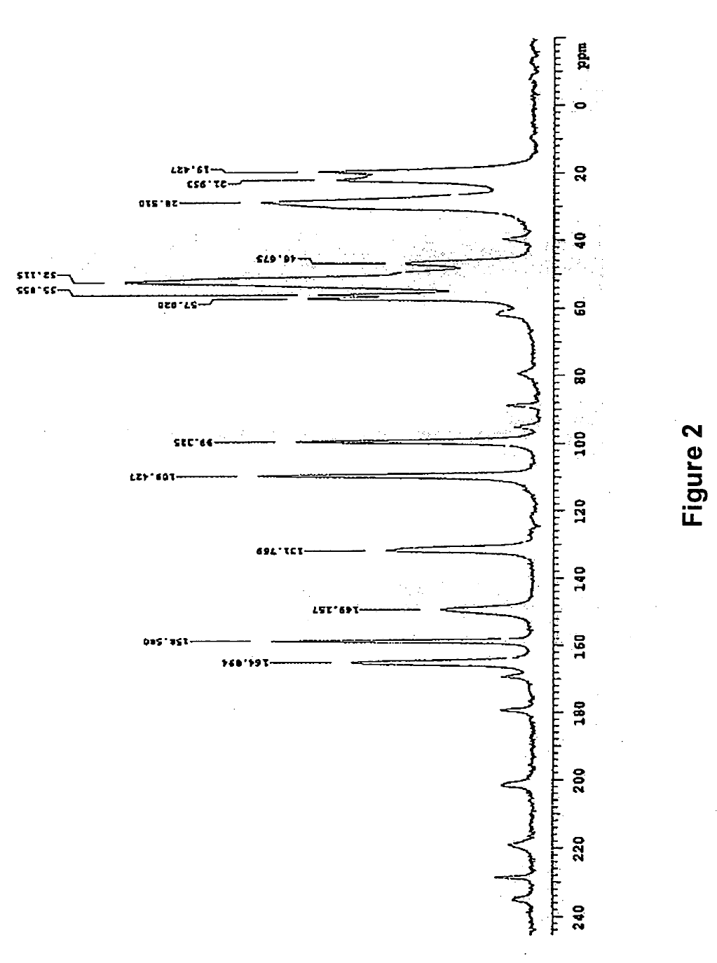

Solid state ¹³C-NMR spectra were collected for Form II and amorphous renzapride hydrochloride hydrate and are illustrated in FIGS. 1 and 2 respectively.

The sample of Form II renzapride hydrochloride hydrate gave a different spectrum to that obtained for amorphous renzapride hydrochloride hydrate. The relaxation properties of Form II renzapride hydrochloride hydrate (which determine the acquisition conditions) also differed to those of amorphous renzapride hydrochloride hydrate. The experimental parameters were therefore optimised for each sample.

Table 1 below sets out the observed signals for Form II renzapride hydrochloride. In addition a proposed assignment of these peaks is provided.

TABLE 1
Solid state ¹³C-NMR data for Form II renzapride hydrochloride hydrate

| Signals (ppm) | Proposed Atom assignment |
|---|---|
| 19.233, 21.856, 28.413, 30.307 | a |
| 46.141, 51.823, 53.329 | b |
| 55.855 | c |
| 99.373 | d |
| 109.184 | e |
| 130.992 | f |
| 149.352 | g |
| 158.483 | h |
| 164.700 | i |
| Not resolved from baseline | j |

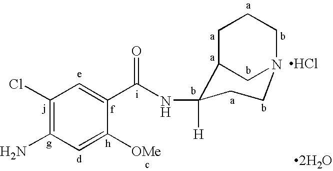

The molecule should give seven high-frequency (90+ ppm) signals (neglecting any fine structure) but only six were detected. The missing signal is from the carbon attached to the chlorine. Coupling between these nuclei results in a broadened signal (and possibly a multiplet) which probably accounts for the signals in the baseline between 100 and 130 ppm. A similar coupling slightly broadens the signals from the carbons attached to nitrogen (for example, the signal at ˜149 ppm is presumably the aromatic C—NH₂).

A proposed assignment of the other signals is as follows: the three —CH₂-s+CH in the group between 15 and 30 ppm, the three —CH₂—N's+>CH—N in the group between 43 and 53 ppm, OMe at 55-58 ppm, amide carbon at 164-168 ppm, aromatic C—O at 156-159 ppm, aromatic C—C at ˜131 ppm and the CH ortho to the OMe is probably the 99 ppm line with the other CH at 109-112 ppm. The remaining unlabelled, low-intensity signals are spinning sidebands and can be ignored.

Comparison of Solid State NMR Spectra of Form II Renzapride Hydrochloride Hydrate and Amorphous Renzapride Hydrochloride Hydrate;

Variation is observed in the pattern of signals observed from 60 to 15 ppm in the spectra obtained for Form II renzapride hydrochloride hydrate and amorphous renzapride hydrochloride hydrate. In addition, the spectrum obtained for amorphous renzapride hydrochloride hydrate contains a signal at 131.8 ppm for the OMe signal which is split. The signal at 131.8 ppm appears to be an unresolved pair. Furthermore the lack of resolution in the low-frequency signals in the amorphous renzapride hydrochloride hydrate spectrum appear to be caused by the presence of additional lines.

### Example 4

### Dynamic Vapour Sorption

Methodology:

Each sample was placed into a sample pan and loaded into the DVS system. An initial weight reading was taken. The sample was then exposed to an atmosphere with a relative humidity (RH) of 0% to dry the sample and a dry weight reading taken. The sample was exposed to an adsorption/desorption cycle between 0 and 90% RH in 10% RH intervals. The change in weight data was fitted to an exponential expression that was used to automatically determine the end point of each stage, which then triggers the next increase/decrease in relative humidity.

For the DVS traces, the stepped lines represent the relative humidity (RH) level in the Chamber. The curved line represents the weight change in the sample. The RH was controlled automatically and changes once the rate of change in weight of the sample is small.

Results: Form II Renzapride Hydrochloride Hydrate:

The sorption isotherm plot for Form II shows a rapid initial uptake in moisture (to ca. 9% w/w) at 10% RH, after which there is very little change in the sample mass, indicating that Form II is a very stable material which is consistent in its properties with respect to moisture.

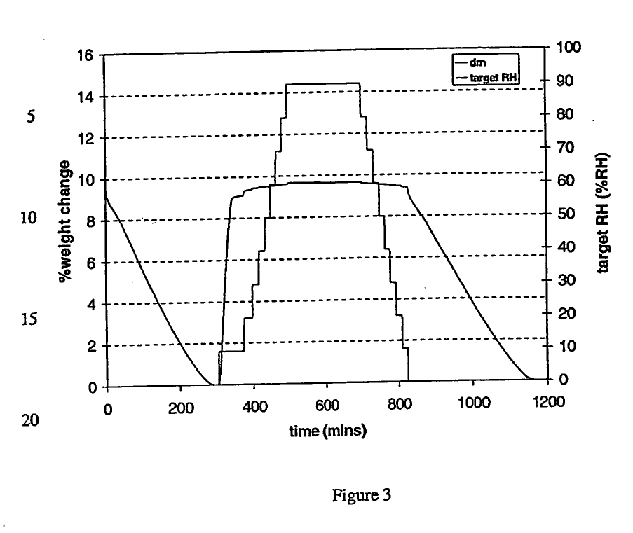

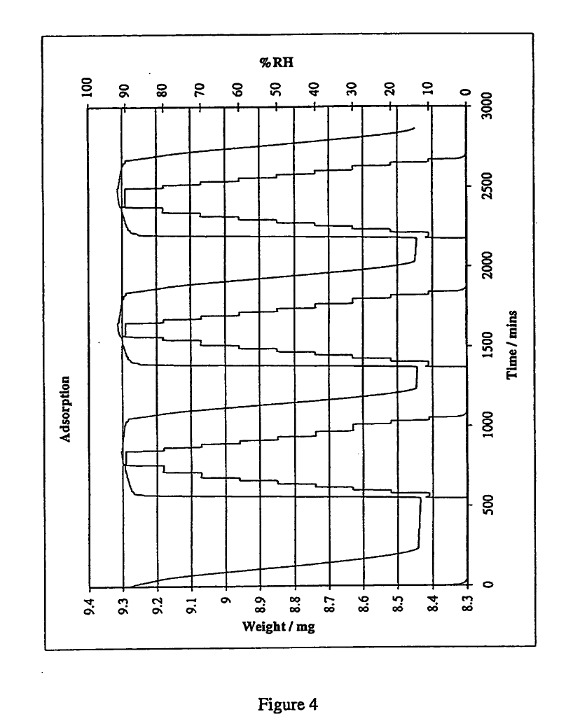

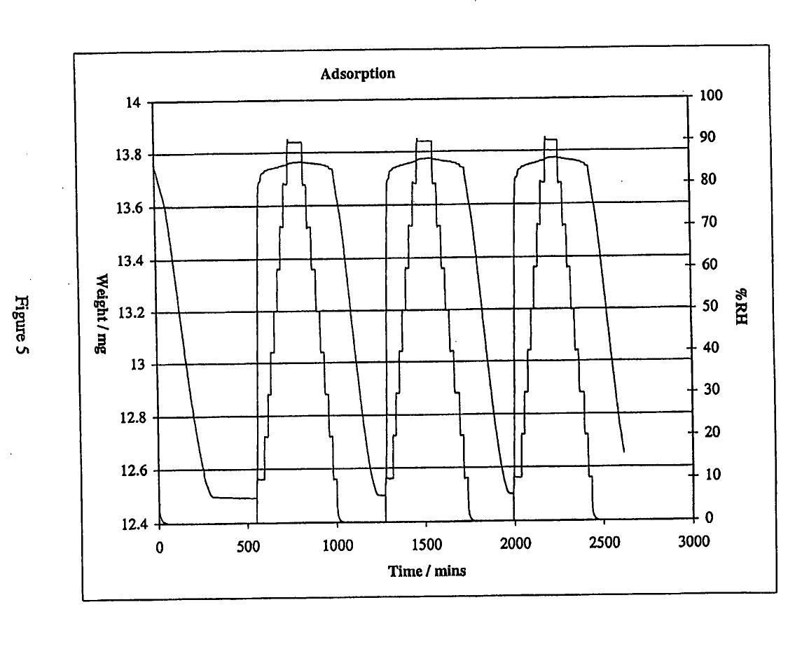

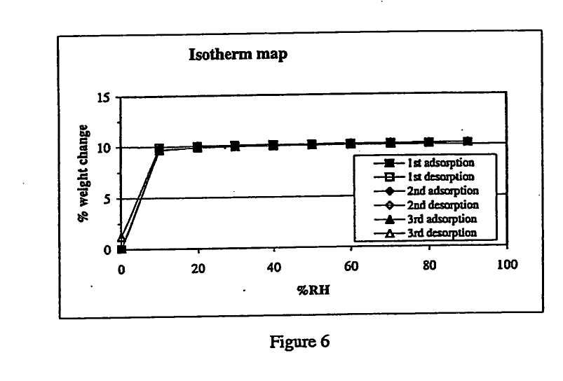

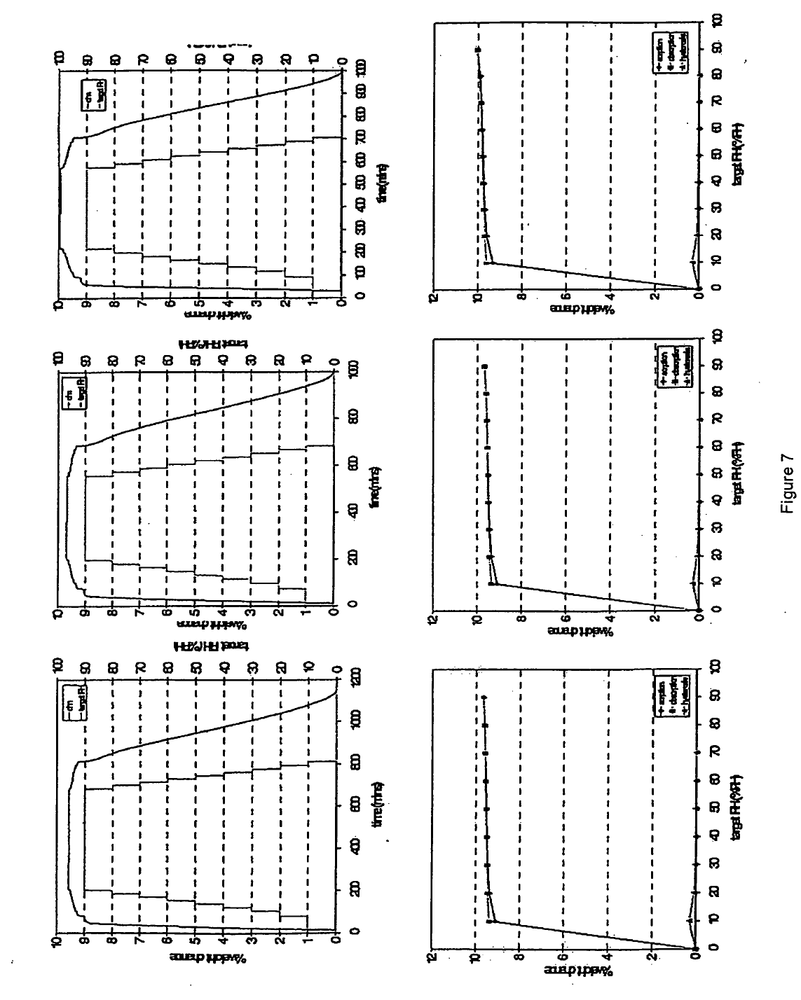

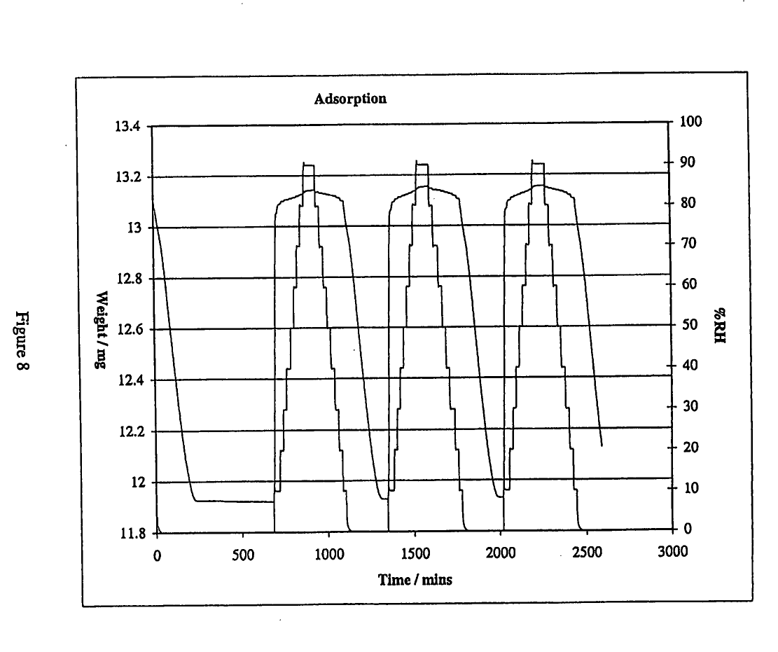

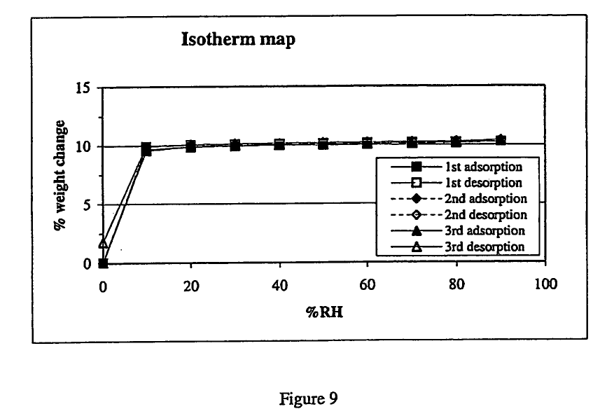

The DVS and isotherm plots for Form II are shown in FIGS. 3 to 9.

Samples of Form II renzapride hydrochloride hydrate show behaviour that implies a very stable material that is consistent in its properties with respect to moisture.

The DVS data provides no evidence of equilibration of Form II renzapride hydrochloride hydrate between different forms. There was very little change in mass when the three cycles are compared with each other (i.e. they are very similar as indicated by the isotherm plots).

In addition there was very little change in the mass of the sample when the chamber had reached 90% RH conditions and the sample had taken up ca 9.5% (w/w) moisture, indicating that the material was stable with respect to moisture content, and consistent across the three cycles. The weight gain observed for Form II renzapride hydrochloride hydrate is consistent with the formation of a dihydrate. The batches analysed all show a very stable profile with respect to exposure to humidity, once above 10% RH. Above 10% RH there is very little variation in the mass of the dihydrate material.

Results: Amorphous Renzapride Hydrochloride Hydrate:

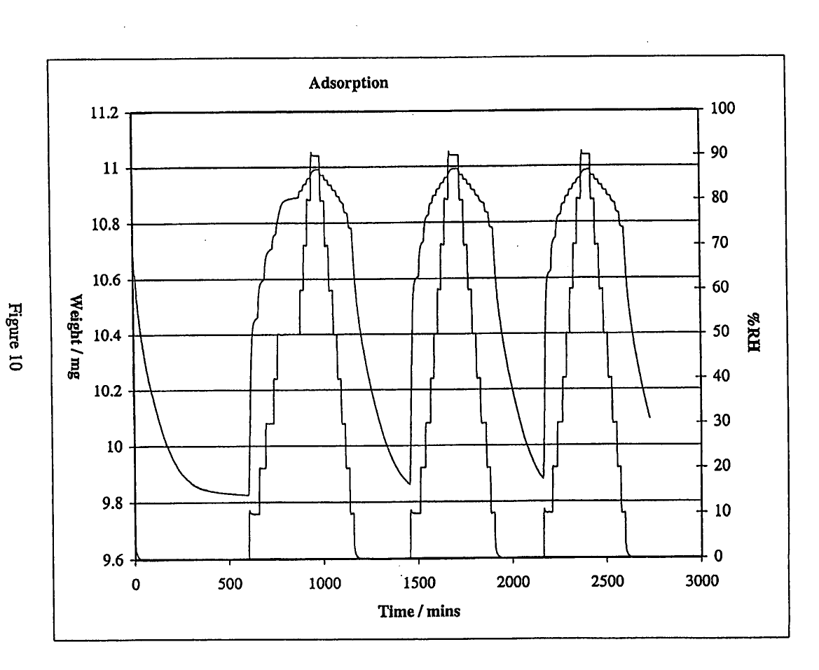

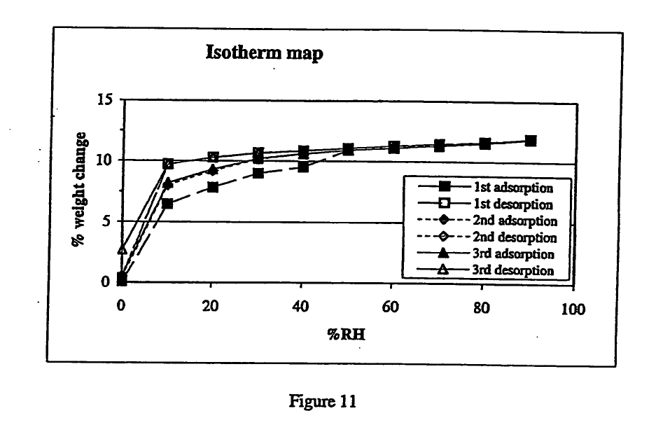

The DVS plot and isotherm map for amorphous renzapride hydrochloride hydrate are shown in FIGS. 10 and 11.

The DVS plot for amorphous renzapride hydrochloride hydrate (FIG. 10) shows approximately 6% (w/w) uptake upon exposure to a 10% RH environment which is followed by a more gradual uptake up to 50% RH. Above this there is a lower uptake of moisture as the RH increases. There is a gradual decrease in mass as the RH decreases from 90 to 10% RH.

The adsorption/desorption profiles for the 2^{nd} and 3^{rd} cycles are identical, with the desorption cycle being identical to that of the 1^{st} cycle. The uptake at 10 to 40% RH is greater than that observed for the 1^{st} cycle and again shows the majority of moisture loss on going from 10 to 0% RH.

The isotherm map (FIG. 11) shows quite clearly the difference between the first and subsequent adsorption cycles. The desorption cycles are identical on all 3 cycles.

The data set in FIGS. 10 and 11 show that there is a clear difference between the sample before and after the first adsorption cycle. There is a significant difference between the first adsorption and desorption cycle. As this cycle is not reproducible, it indicates that a change in the form of the material has occurred, rather than the hysteresis being related to other physical attributes. The overall moisture gain from 0% RH to 90% RH is approximately 12% (w/w), which is higher than the amount required for a stoichiometric dihydrate.

After completion of the first cycle the absorption/desorption profile is reproducible indicating that the material is then stable with respect to its interaction with moisture.

The behaviour of the amorphous renzapride hydrochloride hydrate does not appear to be consistent with that of a dihydrate material.

### Example 5

### Intrinsic Dissolution Rate

Methodology:

The samples were prepared as discs. Each disc was prepared by compressing the sample for 5 minutes at 2 tons of pressure. Each disc was transferred to a static dissolution system, whereby one surface of the disc is exposed to the dissolution medium. Six discs were prepared from each batch of material for duplicate intrinsic dissolution rate (IDR) determinations at pHs 2.2, 4.0 and 7.0. Dissolution samples (0.8 ml) were withdrawn at 5 minute intervals up to 60 minutes and samples were analysed.

The amount of drug released (mg) is divided by the surface area of the disc (0.5 cm²) to obtain an amount per unit area (mg cm⁻²). The average of the duplicate determinations is plotted as a function of time (minutes). The IDR (mg cm⁻² minutes⁻¹) is given by the gradient (determined by linear regression) over the linear range of the release profile. When the dissolution into solution is deemed independent of pH, a pH independent IDR is calculated. The pH independent IDR is calculated by averaging the data for each time point at all of the pHs studied and calculating the linear regression on at least the first 5 data points. All IDR values are reported with the error encompassing the upper and lower 95% confidence limits of the linear regression.

Linear regression analysis of the IDR data used all of the data points, as the release profiles are linear.

TABLE 2
IDR for Form II and amorphous renzapride hydrochloride hydrate
(mg/min/cm²)

| Sample | pH 2.2 | pH 4.0 | pH 7.0 | independent |
|---|---|---|---|---|
| Form II | 5.5 ± 0.2 | 5.7 ± 0.2 | 5.9 ± 0.3 | 5.7 ± 0.3 |
| Amorphous | 6.6 ± 0.2 | 6.7 ± 0.1 | 6.9 ± 0.1 | 6.8 ± 0.3 |

The Form II material exhibits a different intrinsic dissolution rate characteristic to that of the amorphous material.

### Example 6

### Characterisation of Form II Renzapride Hydrochloride Hydrate

Analysis of Moisture Content of Form II Renzapride Hydrochloride Hydrate:

The water content of 11 different samples of Form II renzapride hydrochloride hydrate was determined by Karl Fischer analysis and was reproducibly found to be ca. 9.0% (w/w) water, corresponding to 2 molecules of water being present within the crystalline structure. This reproducibility in the water content of Form II renzapride hydrochloride has been found to be independent of both the method used to produce Form II and the scale of the synthesis. The present invention provides the ability to reproducibly control the amount of water present in Form II renzapride hydrochloride hydrate, which is highly advantageous for controlling the quality of the approved pharmaceutical ingredient during storage, handling, formulation and product manufacture.

Moisture Content (by Karl Fischer):

| | |
|---|---|
| Form II | 9.0% (w/w) ± 0.31 (SD; n = 11) |

Infra Red (IR) Analysis:

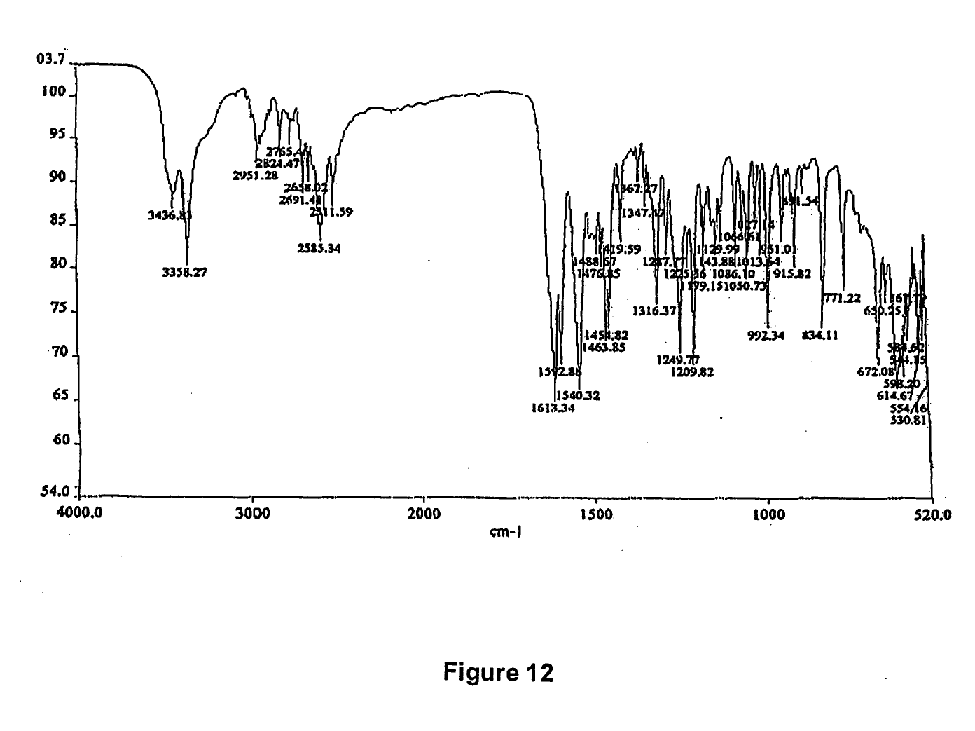

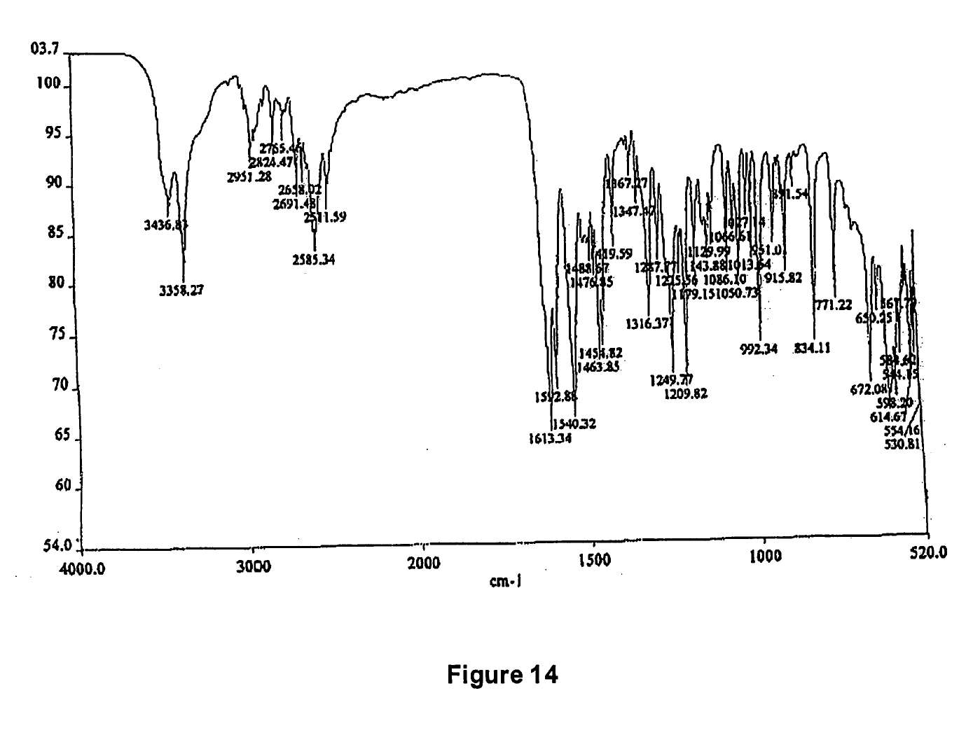

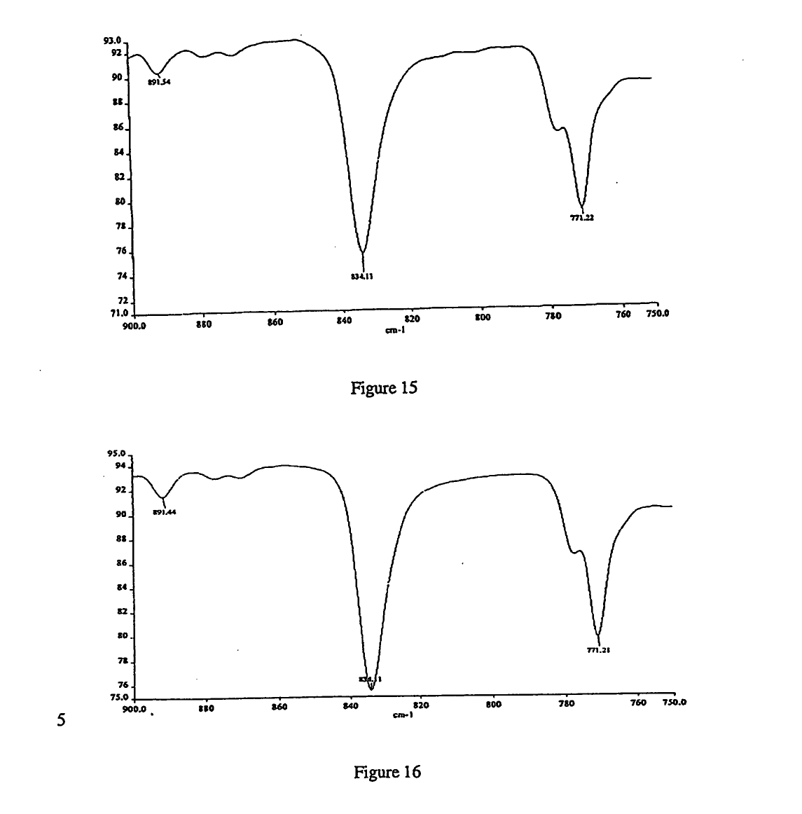

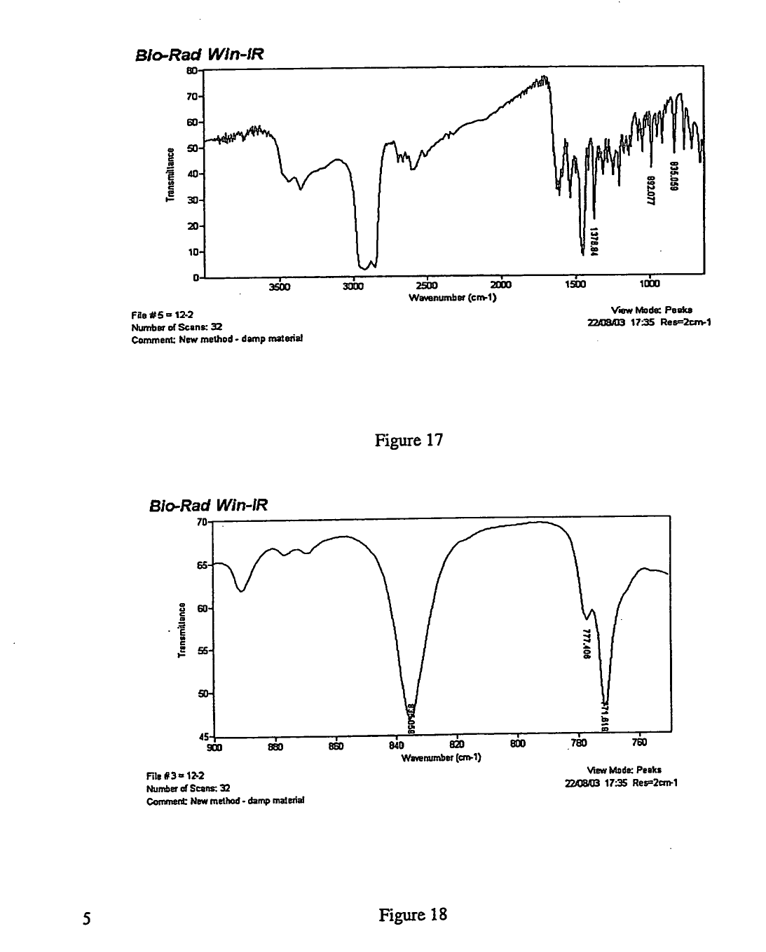

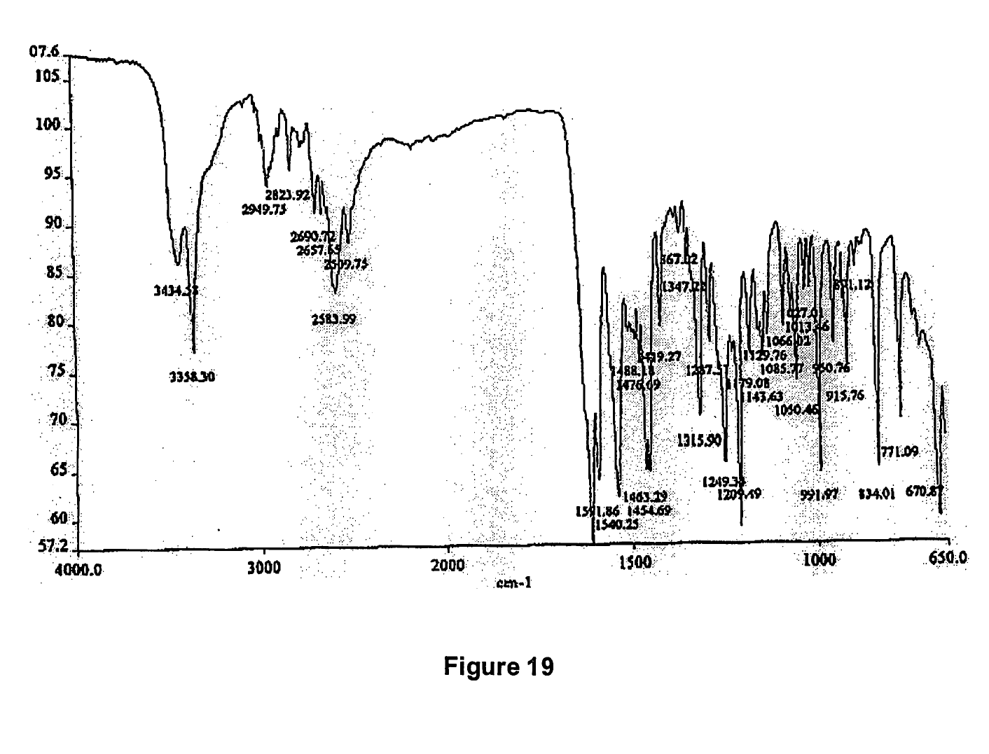

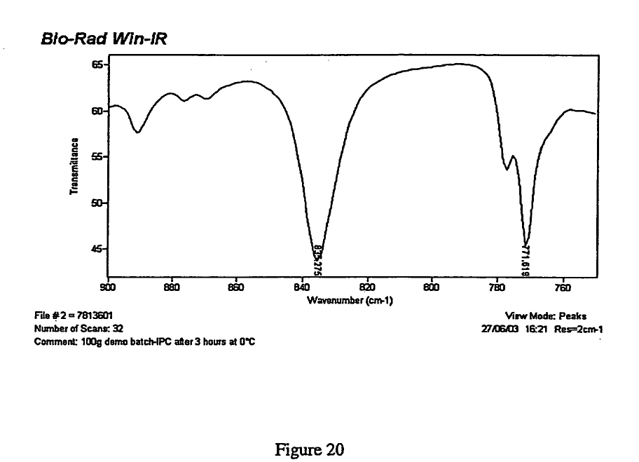

IR spectra were recorded on a number of samples of Form II manufactured according to the processes of the present invention. FIGS. 12 to 20 illustrate the IR spectra of Form II renzapride hydrochloride hydrate. Table 3 below sets out the IR peaks observed wherein each value is in wavenumbers (cm⁻¹). Peak descriptors are s=sharp, br=broad with relative intensities assigned as st=strong, m=medium and w=weak

TABLE 3
Representative IR data for a sample of Form II renzapride
hydrochloride hydrate.

```
Form II
3434.0 (br, m)
3358.5 (s, st)
2951.2 (br, w)
2591.5 (s, w)
2585.4 (br, m)
2511.7 (br, w)
1592.9 (s, st)
1540.8 (s, st)
1463.9 (s, st)
1454.9 (s, st)
1419.6 (s, w)
1316.4 (s, m)
1249.8 (s, m)
1209.9 (s, st)
1143.8 (s, w)
1130.0 (s, w)
1086.1 (s, m)
  992.5 (s, m)
  950.6 (s, w)
  891.4 (s, w)
  834.1 (s, m)
  771.2 (s, w)
  671.8 (s, m)
  650.5 (s, w)
```

All observed samples of Form II renzapride hydrochloride hydrate exhibit a characteristic peak at 835±1.5 cm⁻¹. This peak can be used to distinguish the presence of Form II renzapride hydrochloride hydrate. It will be appreciated that the exact value of this characteristic peak will vary depending on the sample preparation, apparatus used, etc. To this end, Table 4 sets out a number of values for this characteristic peak as observed for different samples of Form II renzapride hydrochloride hydrate.

The range for the signature peak in the IR spectrum for Form II is derived from the values tabulated below in Table 4.

TABLE 4
Values observed for characteristic peak at 835 + 1.5 cm⁻¹

| Form II Sample | Signature peak (cm⁻¹) |
|---|---|
| 1 | 834.11 |
| 2 | 834.11 |
| 3 | 835.06 |
| 4 | 834.31 |
| 5 | 834.31 |
| 6 | 834.91 |
| 7 | 834.01, 835.29 and 834.12 on repeat runs |
| 8 | 834.31 |
| 9 | 835.28 |
| 10 | 835.28 |
| 11 | 835.28 |
| 12 | 836.28 |

The presence of a characteristic peak at 835±1.5 cm⁻¹ in the IR spectra of Form II renzapride hydrochloride hydrate permits confirmation that Form II renzapride hydrochloride hydrate is present.

XRPD Analysis of Form II Renzapride Hydrochloride:

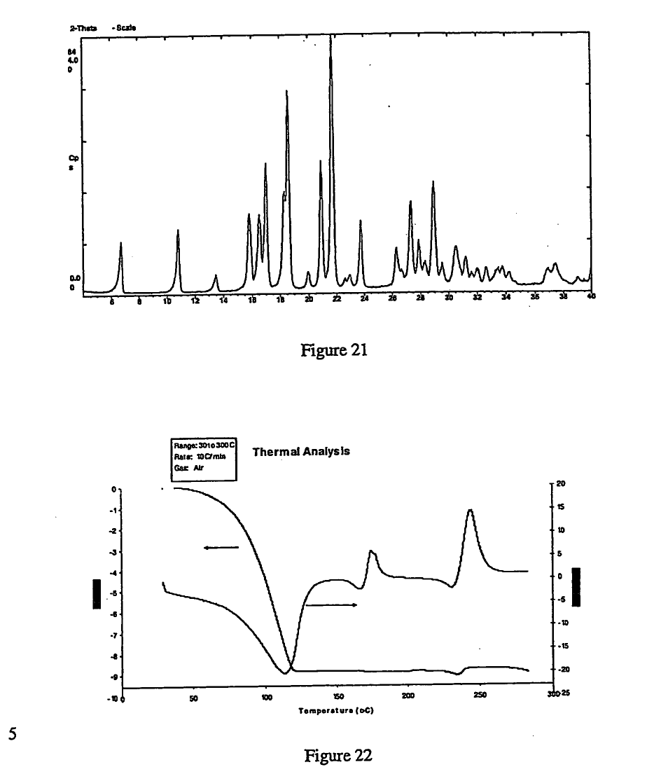

Form II renzapride hydrochloride hydrate has been analysed by X-ray Powder Diffraction: The X-ray powder diffraction (XRPD) pattern of Form II renzapride hydrochloride hydrate is shown in FIG. 21. The presence of defined peaks in the XRPD pattern indicates that Form II has a crystalline character.

Stability of Form II Renzapride Hydrochloride Hydrate:

Samples of Form II renzapride hydrochloride hydrate were placed in storage under conditions of 25° C./60% relative humidity. Form II renzapride hydrochloride hydrate shows good stability to moisture on storage as shown in Table 5, below.

TABLE 5
Stability of Form II renzapride hydrochloride hydrate

| | t = 0 | t = 6 | t = 12 | t = 18 | t = 24 |
|---|---|---|---|---|---|
| Stability test | months | months | months | months | months |
| 1. Description | White | White | White | White | White |
| | uniform | uniform | uniform | uniform | uniform |
| | solid | solid | solid | solid | solid |
| 2. Water | 8.9%(w/w) | 8.6%(w/w) | 8.8%(w/w) | 8.8%(w/w) | 8.8%(w/w) |
| content | | | | | |
| (by Karl | | | | | |
| Fisher) | | | | | |

Particle Size Distribution:

The Particle Size Distribution (PDS) of renzapride is measured according to the method detailed below.

| | |
|---|---|
| Material Parameters: | renzapride: R.I. = 1.5 Absorption = 0.1 |
| | Model: General purpose (fine)-enhanced sensitivity |
| Measurement | Measurement time: 4 secs |
| Parameters: | Background time: 8 secs |
| | Obscuration limits: 3 < Obscuration < 20 (filter on) |
| Sampling | Scirocco 2000 |
| Parameters: | |

Vibration feed rate: 40%
Air pressure: 3.5 Bar

Single measurement of each batch performed in triplicate.

TABLE 6
Particle Size Distribution of Form II renzapride hydrochloride
hydrate manufactured as a 5 kg batch.

| | d (0.1)/μm | d(0.5)/μm | d(0.9)/μm | Span* |
|---|---|---|---|---|
| Form II | 11.5 | 47.1 | 109.0 | 2.1 |

*Span = (d(0.9) − d(0.1))/d(0.5)

As shown in Table 6, above, Form II shows a narrow particle size distribution, with a span of 2.1.

Photomicroscopy:

A small amount of sample was dispersed in silicon oil (on a microscope slide) and a cover slip place over it. Images of the sample were captured (minimum of two magnifications) using calibrated image capture software.

Form II renzapride hydrochloride hydrate is composed of regular shaped cubic/rhombohedra crystals that are preferred for the manufacture of a solid dosage form.

Thermochemical Analysis:

A TGA trace of the Form II is illustrated in FIG. 22. The TGA trace for Form II renzapride hydrochloride hydrate contains an endotherm at 150-180° C. This endotherm appears to be associated with a phase change of the dehydrated material, following loss of water during analysis.

The thermogram illustrates a large endothermic peak (with Tₘₐₓ at approximately 100° C.) due to the loss of two moles of water. The endothermic peak is followed by two exothermic peaks at Tₘₐₓ 170° C. and 240° C. The Form II material therefore undergoes loss of water to a dehydrated hydrate, which at around 150-170° C. undergoes a phase change that then proceeds to melt around 270° C. and finally decomposes at elevated temperatures.

Solubility Studies:

Solubility studies on Form II renzapride hydrochloride hydrate in various water/solvent mixtures were carried out using the following standard procedure:

Approximately 50-100 mg of Form II renzapride hydrochloride hydrate was added to a 10 cm³ glass vial and 5 cm³ of the appropriate solvent was added. The suspensions were stirred at 20° C. for 24 hours. After this time the suspensions were filtered and the clear solutions were analysed by HPLC. The results are summarised below in Table 7.

TABLE 7
Solubility studies of Form II renzapride hydrochloride hydrate

| Water/Solvent Mixtures | Solubility (mg/mL) |
|---|---|
| 1% water in ethanol | 9.0 |
| 5% water in ethanol | 17.5 |
| 10% water in ethanol | 21.2 |
| 1% water in IPA | 1.1 |
| 5% water in IPA | 2.8 |
| 10% water in IPA | 4.6 |
| 1% water in acetone | 0 |
| 5% water in acetone | 0.9 |
| 10% water in acetone | 6.7 |
| 1% water in THF | 0 |
| 5% water in THF | 0.4 |
| 10% water in THF | 8.9 |

### Example 7

### Removal of Ethanol by Solvent Slurry

Residual solvent can be removed from Form II by solvent slurry. A series of slurries were performed in water-wet acetone, THF, TBME and toluene to investigate the effect on the level of ethanol. The following standard procedure was used:

100 mg of Form II was stirred at room temperature in 1 cm³ (10 vol) of solvent for 24 hours. After this time the solid was isolated by filtration and analysed for solvent content and physical form. The results are summarised in Tables 8 and 9:

TABLE 8
Removal of ethanol by solvent slurry

| Solvent | Solvent content | Ethanol content |
|---|---|---|
| 5% water in THF | Not detected | Not detected |
| Wet TBME | Trace (0.1% w/w) | Not detected |
| Wet Toluene | Not detected | Not detected |

The results show that ethanol can be removed by solvent slurries in acetone, tetrahydrofuran (THF), toluene and TBME to a level not detected by ¹H NMR (estimated to be <0.1% detection). The form of the material isolated from the slurries was determined by IR and found to be Form II in all cases.

TABLE 9
Isolated form after solvent slurry

| Solvent | Isolated form |
|---|---|
| 5% water in acetone | Form II |
| 5% water in THF | Form II |
| Wet TBME | Form II |
| Wet Toluene | Form II |

### Example 8

### Crystallisation and Drying Studies With Form II

In order to remove any residual ethanol, the material is dried under vacuum at up to 80° C. The ‘ethanol-free’ solid is then rehydrated to provide the final material.

To investigate the drying of different forms of renzapride hydrochloride hydrate, Ig samples were prepared from crystallisations in 1% water in ethanol, 5% water in ethanol, 1% water in IPA and 5% water in IPA. It will be appreciated that the recrystallisation procedure set out below allows the reworking of renzapride hydrochloride hydrate to Form II renzapride hydrochloride hydrate. The following procedure was used:

1 g of renzapride hydrochloride hydrate was dissolved in the minimum amount of refluxing solvent and then allowed to cool for ca. 1 hour at room temperature and then placed at 5° C. for a further 18 hours. Solid was isolated by filtration and analysed by NMR, IR and KF. The data obtained is summarised in Tables 10 and 11 below.

TABLE 10
Initial Analysis

| | | | Solvent | | |
|---|---|---|---|---|---|
| | Water | Recovery* | content^{#} | Water (KF)^{#} | |
| Solvent | content | (mg) | % (w/w) | % (w/w) | Form (by IR)^{#} |
| Ethanol | 1% | 599 | 5.37 | 1.73 ± 0.29 | Other |
| | 5% | 714 | 0.16 | 9.21 ± 0.42 | Form II |
| IPA | 1% | 565 | 9.07 | 0.99 ± 0.05 | Other |
| | 5% | 698 | 0.65 | 8.96 ± 0.34 | Form II |

*Crude weight - not corrected for solvent or water
^{#}after drying under vacuum (17 mbar) at 30° C. for 4 hours

TABLE 11
After Further Drying

| | | Solvent content^{#} | Water (KF)^{#} |
|---|---|---|---|
| Solvent | Water content | % (w/w) | % (w/w) |
| Ethanol | 1% | 0.16^{a} | 1.08 ± 0.5 |
| | 5% | 0.02 | 0.99 ± 0.4 |
| IPA | 1% | 1.20 | 0.61 ± 0.02 |
| | 5% | 0.15 | 0.57 ± 0.1 |

^{a}the solvent was apparently IPA, presumably due to drying the samples in the oven with the IPA derived material
^{#}after drying under vacuum (17 mbar) at 30° C. for 4 hours + 15 hours at 60° C.

Initial analysis of the samples shows that Form II (containing ca. 9% water) was produced by crystallisation in either 5% water in ethanol or 5% water in IPA. The isolated material initially contained relatively low solvent residues, 0.16% ethanol and 0.65% IPA, respectively. These solvent levels were much lower than those found for the material isolated from either 1% water in ethanol or 1% water in IPA, which contain 5.4% and 9.1%, respectively. This suggests that Form II dihydrate material does not hold onto excess solvent readily and as a consequence will be easier to dry than the material isolated from solvents containing low water levels (<4%).

Further drying of the samples shows that after heating for 60° C. under vacuum (17 mbar) solvent and water have been removed from all the samples.

### Example 9

### Summary of Characterisation of Form II

The characterisation data for Form II renzapride hydrochloride hydrate indicates that it is a crystalline material that gives a sharp X-ray diffraction pattern and IR spectrum and has a well-defined water content. The water content does not change over a wide range of humidity as indicated by DVS experiments. This allows long term storage of the material. Form II has a particle size distribution with a consistently narrow span, with satisfactory blend homogeneities. Microscopy has shown the batches to have consistent regular shaped crystals that are ideal for solid dosage form manufacture. Form II will behave in a predictable fashion during dispensing and manufacture. Form II has a diagnostic peak in the IR at 835±1.5 cm⁻¹ (sharp) that can be used to identify the presence of the form.

According to another embodiments, the present invention provides Form II renzapride hydrochloride hydrate, isolated from any other the methods described above and herein, having at least one feature selected from the group consisting of:
(a) a characteristic peak in its Infra-Red spectrum at 835±1.5 cm⁻¹;
(b) an X-ray powder diffraction pattern substantially as depicted in FIG. 21;
(c) 8.3 to 9.8% water content; and
(d) a solid state ¹³C NMR spectrum substantially as depicted in FIG. 1.

According to yet another embodiments, the present invention provides Form II renzapride hydrochloride hydrate, isolated from any other the methods described above and herein, having at least one feature selected from the group consisting of:
(a) a characteristic peak in its Infra-Red spectrum at 835±1.5 cm⁻¹;
(b) an X-ray powder diffraction pattern substantially as depicted in FIG. 21; and
(c) 8.3 to 9.8% water content.

Another aspect of the present invention provices crystalline Form II renzapride hydrochloride hydrate having at least one feature selected from the group consisting of:
(a) a characteristic peak in its Infra-Red spectrum at 835±1.5 cm⁻¹;
(b) an X-ray powder diffraction pattern substantially as depicted in FIG. 21;
(c) 8.3 to 9.8% water content; and
(d) a solid state ¹³C NMR spectrum substantially as depicted in FIG. 1.

Yet another aspect of the present invention provices crystalline Form II renzapride hydrochloride hydrate having at least one feature selected from the group consisting of:
(a) a characteristic peak in its Infra-Red spectrum at 835±1.5 cm⁻¹;
(b) an X-ray powder diffraction pattern substantially as depicted in FIG. 21; and
(c) 8.3 to 9.8% water content.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method for preparing Form II renzapride hydrochloride hydrate comprising the steps of:
(a) providing renzapride;
(b) incubating renzapride in a solution of water and a water miscible solvent;
(c) adding concentrated hydrochloric acid to the renzapride solution; and
(d) isolating the resulting Form II renzapride hydrochloride hydrate.

2. The method according to claim 1, wherein the water miscible solvent is one or more of THF, acetone or an alcohol.

3. The method according to claim 2, wherein the alcohol is one or more of methanol, ethanol, n-propanol, isopropanol, n-butanol or tert-butanol.

4. The method according to claim 1, wherein the water and water miscible solvent solution contains from 3% to 15% water.

5. The method according to claim 1, wherein the renzapride is prepared by the deprotection of compound (8):

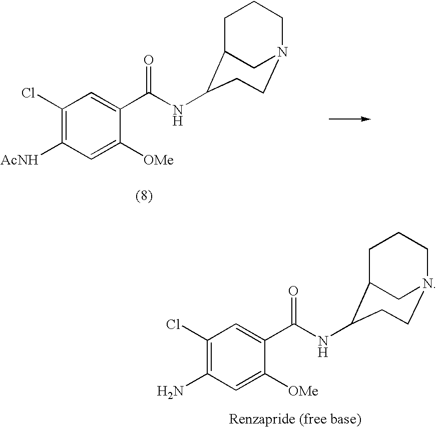

6. The method according to claim 1, wherein the Form II renzapride hydrochloride hydrate isolated at step (d) has at least one feature selected from the group consisting of:
(a) a characteristic peak in its Infra-Red spectrum at 835±1.5 cm⁻¹;
(b) an X-ray powder diffraction pattern substantially as depicted in FIG. 21; and
(c) 8.3 to 9.8% water content.

7. A method for preparing Form II renzapride hydrochloride hydrate from renzapride hydrochloride, said process comprising the steps of:
(a) forming a saturated solution of renzapride hydrochloride in a solvent system comprising an organic solvent and from 3% to 30% water; and
(b) isolating Form II renzapride hydrochloride hydrate therefrom.

8. The method according to claim 7, wherein the Form II renzapride hydrochloride hydrate is isolated by crystallisation.

9. The method according to claim 7, wherein the solvent system in step (a) comprises one or more solvents which can solubilize renzapride hydrochloride and which are miscible with water.

10. The method according to claim 7, wherein the solvent is one or more of ethanol, acetone, isopropyl alcohol, TBME, or THF.

11. The method according to claim 8, wherein the recrystallisation of renzapride hydrochloride hydrate is carried out in an aqueous ethanol solution.

12. The method according to claim 7, wherein the Form II renzapride hydrochloride hydrate isolated at step (b) has at least one feature selected from the group consisting of:
(a) a characteristic peak in its Infra-Red spectrum at 835±1.5 cm⁻¹;
(b) an X-ray powder diffraction pattern substantially as depicted in FIG. 21; and
(c) 8.3 to 9.8% water content.

13. A method of preparing Form II renzapride hydrochloride hydrate comprising the steps of:
(a) slurrying renzapride hydrochloride in an organic solvent comprising 4 to 25% water; and
(b) isolating Form II renzapride hydrochloride hydrate therefrom.

14. The method according to claim 13, wherein the organic solvent is one or more of ethanol, acetone, isopropyl alcohol, TBME or THF.

15. The method according to claim 14, wherein the organic solvent comprises 6 to 10% water.

16. The method according to claim 13, wherein the Form II renzapride hydrochloride hydrate isolated at step (b) has at least one feature selected from the group consisting of:
(a) a characteristic peak in its Infra-Red spectrum at 835±1.5 cm⁻¹;
(b) an X-ray powder diffraction pattern substantially as depicted in FIG. 21; and
(c) 8.3 to 9.8% water content.

17. Form II renzapride hydrochloride hydrate as produced by any one of claims 1 to 16.

18. Crystalline Form II renzapride hydrochloride hydrate comprising two moles of water per mole of renzapride hydrochloride at a level of 75% Form II or above.

19. The crystalline Form II renzapride hydrochloride hydrate according to claim 18, containing from 8.3 to 9.8% water.

20. The crystalline Form II renzapride hydrochloride hydrate according to either of claims 18 or 19 having a characteristic peak in its Infra-Red spectrum at 835±1.5 cm⁻¹.

21. The crystalline Form II renzapride hydrochloride hydrate according to claim 18, having an Infra-Red spectrum substantially as depicted in FIG. 12, 14, 17 or 19.

22. The crystalline Form II renzapride hydrochloride hydrate according to claim 18, having an Infra-Red spectrum with characteristics sufficient to distinguish said Form II from other forms of renzapride hydrochloride hydrate.

23. A method of identifying crystalline Form II renzapride hydrochloride hydrate in a sample comprising the steps of carrying out infra-red spectroscopy on a sample of renzapride hydrochloride hydrate and monitoring for the presence of a characteristic peak at 835±1.5 cm⁻¹.

24. The crystalline Form II renzapride hydrochloride hydrate according to claim 18, wherein said Form II has at least one feature selected from the group consisting of:
(a) a characteristic peak in its Infra-Red spectrum at 835±1.5 cm⁻¹;
(b) an X-ray powder diffraction pattern substantially as depicted in FIG. 21; and
(c) 8.3 to 9.8% water content.

25. A composition comprising Form II renzapride hydrochloride hydrate according to claim 18, and a pharmaceutically acceptable carrier, adjuvant, or vehicle.

26. A method of treating, preventing, or lessening the severity of one or more conditions, disorders, or symptoms selected from irritable bowel syndrome, retarded or delayed gastric emptying, dyspepsia, oesophageal reflux, peptic ulcer, flatulence, impaired evacuation, constipation, diabetic neuropathy, functional abdominal bloating, abdominal pain or discomfort, abdominal bloating, an abnormality in stool consistency, an abnormality in frequency of stool passage, a feeling of incomplete emptying, feelings of urgency, passage of mucus, emesis, gastroparesis or a disorder of the central nervous system, wherein said method comprises administering to a patient in need thereof a composition according to claim 25.

27. The method according to claim 26, wherein said condition, disorder, or symptom is constipation-predominant, diarrhoea-predominant or alternating (mixed-symptom) irritable bowel syndrome.

28. The method according to claim 26, wherein said condition, disorder, or symptom is irritable bowel syndrome (IBS), constipation, gastroparesis and abdominal pain and discomfort.

29. A method of treating, preventing, or lessening the severity of a disorder relating to impaired gastro-intestinal motility, comprising administering to a patient in need thereof a composition according to claim 25.
